SEQUENCE LISTING (1 sequence)

SEQ ID NO: 1 - DNA, 13 bp, Artificial Sequence
  Synthetic
aacgaggcgcacc

(19) United States
(12) Patent Application Publication
(10) Pub. No.: US 2007/0049745 A1
(43) Pub. Date: Mar. 1, 2007

(54) Oligonucleotide synthesis using periodate salts

(75) Inventor: Zbigniev Skrzypczynski, Verona, WI (US)

Correspondence Address: MEDLEN & CARROLL, LLP, 101 HOWARD STREET, SUITE 350, SAN FRANCISCO, CA, 94105, US

(73) Assignee: Third Wave Technologies, Inc., Madison, WI (US)

(21) Appl. No.: 11/484,817

(22) Filed: Jul. 11, 2006

Related U.S. Application Data
(60) Provisional application No. 60/698,166, filed on Jul. 11, 2005.

(51) Int. Cl. C07H 21/04

(52) U.S. Cl. 536/25.34

(57) ABSTRACT

The present invention relates generally to nucleic acid chemistry and to the chemical synthesis of oligonucleotides. More particularly, the invention relates to improved methods for synthesizing oligonucleotides wherein periodate salts are used (e.g., in organic solvents) as an oxidation reagent in oligonucleotide synthesis (e.g., for automated phosphoramidite synthesis of oligonucleotides). The invention finds utility in the fields of biochemistry, molecular biology, and pharmacology, and in medical diagnostic and screening technologies.

The present invention claims priority to U.S. Provisional Patent Application No. 60/698,166, filed Jul. 11, 2005, hereby incorporated by reference in its entirety.

### FIELD OF THE INVENTION

The present invention relates generally to nucleic acid chemistry and to the chemical synthesis of oligonucleotides. More particularly, the invention relates to improved methods for synthesizing oligonucleotides wherein periodate salts are used (e.g., in organic solvents) as an oxidation reagent in oligonucleotide synthesis (e.g., for automated phosphoramidite synthesis of oligonucleotides). The invention finds utility in the fields of biochemistry, molecular biology, and pharmacology, and in medical diagnostic and screening technologies.

### BACKGROUND OF THE INVENTION

Chemical synthesis of DNA fragments (e.g., solid phase synthesis) is routinely performed using protected nucleoside phosphoramidites (See, e.g., Beaucage et al. 1981, Tetrahedron Lett. 22:1859). Generally, in this approach the 3′-hydroxyl group of an initially 5′-protected nucleoside is first covalently attached to a solid support (e.g., a polymeric support; See, e.g., Pless et al. 1975, Nucleic Acids Res. 2:773). Synthesis of the oligonucleotide then proceeds by deprotection of the 5′-hydroxyl group of the attached nucleoside, followed by coupling of an incoming nucleoside-3′-phosphoramidite to the deprotected hydroxyl group (See, e.g., Matteucci et al. 1981, J. Am. Chem. Soc. 103:3185). The resulting phosphite triester is finally oxidized to a phosphorotriester to complete the internucleotide bond (i.e., to make a more stable structure; See, e.g., Letsinger et al. 1976, J. Am. Chem. Soc. 98:3655). The steps of deprotection, coupling, capping unreacted 5′ OH groups and oxidation are repeated until an oligonucleotide of the desired length and sequence is obtained.

Since its introduction, the phosphoramidite protocol of automated solid phase oligonucleotide synthesis has become widely accepted (See, e.g., Letsinger and Lunsdorf, J. Am. Chem. Soc. 1976, 98,3655-3661; Matteucci and Caruthers, J Am. Chem. Soc., 1981, 103, 3186-3191; Beacage and Iyer, Tetrahedron, 1992, 48,2223-2311) as the method of choice for the preparation of both natural and modified DNA probes (See, e.g., Tsukamo and Hayakawa, in Frontiers in Organic Chemistry, Atta-ur-Rahman (Ed.); 2005; Bentham Science Publisher, Vol 1, pp 3-40). In general, as mentioned above, there are four synthetic steps of a phophoramidite synthesis protocol. These include detritylation, coupling, oxidation and capping. Among these synthetic steps, oxidation is one of the most important. This step converts the acid sensitive internucleotide phosphite triester linkage into a stable phosphate linkage, which makes possible the acid promoted removal of the protecting group (e.g., 5′-dimethoxutrityl (DMT)) and subsequent coupling with the appropriate phosphoramidite reagent.

Currently, most phosphoramidite coupling protocols utilize iodine in THF/water/pyridine solution as the reagent of choice for oxidation of the unstable phosphite group. In spite of its popularity, the use of this reagent often leads to unwanted side effects, particularly in cases when water, base, or iodine sensitive groups (e.g., linkers, dyes, labels, etc.) are present in the structure of the chemically synthesized oligonucleotide molecule. Recent literature describes several reagents that can be used for oxidation of the phosphite internucleotide bond under nonbasic and non-aqueous conditions (See, Sierzchala et al., J. Am. Chem. Soc., 125, 13427-13441 (2003); Uzagare et al., Bioorganic & Medicinal Chemistry Letters, 13, 3537-3540 (2003); Kataoka et al., Organic Letters, 3, 815-818 (2001); Manoharan et al., Organic Letters, 2, 243-246 (2000)). However, these reagents suffer from high cost, toxicity, danger of explosion or lack of commercial availability. Furthermore, in cases of more aggressive oxidizers, the occurrence of undesired oxidative modification of the nucleobases was reported.

Thus, a need exists for new oxidizing reagents compatible with phosphoramidite oligonucleotide synthesis that can be used to oxidize and stabilize the internucleotide phosphite triester linkage into a stable phosphate linkage. Such reagents should be inexpensive, easily obtainable and able to be used under non-aqueous conditions. For example, these reagents should be stable and soluble in organic solvents.

### SUMMARY OF THE INVENTION

The present invention relates to improved methods for synthesizing oligonucleotides wherein periodate salts are used (e.g., in organic solvents) as an oxidation reagent in oligonucleotide synthesis (e.g., for automated phosphoramidite synthesis of oligonucleotides). The invention finds utility in the fields of biochemistry, molecular biology, and pharmacology, and in medical diagnostic and screening technologies.

Accordingly, the present invention provides a method for synthesizing oligonucleotides, comprising removing a protecting group from the 5′ or 3′ carbon of the pentose sugar of a recipient nucleotide; thereby leaving a reactive hydroxyl; coupling a phosphoramidite monomer to the reactive hydroxyl; capping unreacted hydroxyls; and stabilizing the phosphate linkage between the growing oligonucleotide chain and the most recently added base; wherein a reagent comprising a periodate salt is used for the stabilizing. Oligonucleotides synthesized using the compositions and methods of the present invention find use in a broad range of applications, including, but not limited to, polymerase chain reaction, probes, primers, microarrays, siRNAs, RNAi, gene silencing, diagnostics (e.g., medical diagnostics including genotyping), INVADER assays, and other molecular biological techniques. In some embodiments, the stabilizing comprises converting an acid sensitive internucleotide phosphite triester linkage into a stable phosphate linkage. In some embodiments, the protecting group comprises a trityl group. In some embodiments, the trityl group is dimethoxytrityl. In some embodiments, the periodate salt is present within an organic solvent. In some embodiments, the protecting group is removed via exposure to acid. In some embodiments, the acid is trichloroacetic acid (TCA). The present invention is not limited by the type of periodate salt utilized. In some embodiments, the periodate salt comprises tetrabutylammonium periodate and/or phosphonium periodate. In some embodiments, the phosphonium periodate is benzyltriphenylphosphonium periodate. In some embodiments, reagents (e.g., counter-cations) that increase the solubility of the periodate salts in organic solvents are provided. In some embodiments, periodate salts are supported on solid supports (e.g., resins, polymers, etc.) as reagents for solution-phase organic synthesis of biomolecules (e.g., oligonucleotides.) In some embodiments, the organic solvent comprises methylene chloride and/or acetonitrile. In some embodiments, the oligonucleotides comprise a detectable label. In some embodiments, the detectable label comprises a fluorescent molecule. In some embodiments, the fluorescent molecule comprises fluorescein. The present invention is not limited to any particular label. Indeed a variety of detectable labels are contemplated to be useful in the present invention, including any “reporter molecule” that is detectable in any detection system, including, but not limited to enzyme, fluorescent, radioactive, and luminescent molecules and systems. It is not intended that the present invention be limited to any particular detection system or label. In some embodiments, synthesizing oligonucleotides comprises solid phase synthesis. In some embodiments, solid phase synthesis comprises a nucleoside bound to a support through its 5′-hydroxyl group. The present invention is not limited to any particular solid support. Indeed a variety of supports are contemplated to be useful in the present invention, including, but not limited to, solid substrate having a surface to which chemical entities may bind. Suitable solid supports are typically polymeric, and may have a variety of forms and compositions and may derive from naturally occurring materials, naturally occurring materials that have been synthetically modified, or synthetic materials. Examples of suitable support materials include, but are not limited to, polysaccharides such as agarose, dextran, polyacrylamides, polystyrenes, polyvinyl alcohols, polyethylene glycols (PEG), copolymers of hydroxyethyl methacrylate and methyl methacrylate, silicas, teflons, glasses, and the like. In some embodiments, the initial monomer of the oligonucleotide to be synthesized on the substrate surface is bound to a linking moiety. In some embodiments, the linking moiety is bound to a surface hydrophilic group (e.g., to a surface hydroxyl moiety present on a silica substrate).

The present invention also provides a method for preparing an oligonucleotide for acid-based removal of a protecting group comprising treating the oligonucleotide with a reagent comprising a periodate salt. In some embodiments, the reagent comprising a periodate salt converts an acid sensitive internucleotide phosphite triester linkage into a stable phosphate linkage. In some embodiments, the reagent comprising a periodate salt is applied during each cycle of oligonucleotide synthesis.

The present invention also provides a kit comprising a periodate salt and a nucleotide. In some embodiments, the kit further comprises a reagent for nucleic acid synthesis selected from the group consisting of, but not limited to, TCA, phosphoramide monomer, tetrazol, control pure glass bead, 5′ DMT protecting group, and a microarray plate.

The present invention also provides phosphonium periodate supported on a solid support for use in solid phase synthesis ( e.g. organic combinatorial synthesis). The present invention is not limited to any particular solid support. Indeed, a variety of solid supports are contemplated to be useful in the present invention including, but not limited to, resins, CPG, and like polymers. In some embodiments, a polymer supported periodate is a reagent used for liquid-phase organic synthesis ( e.g., organic combinatorial synthesis), including, but not limited to, those that utilize polymer supported reagents.

### DESCRIPTION OF THE DRAWINGS

FIG. 1 shows a synthesis scheme of phosphonium periodate 1.

FIG. 2 shows oxidation of the internucleotide phosphite 2 bond by the periodate solution.

FIG. 3 shows a comparison of RP HPLC profiles of crude reaction products 4a, 4b and 4c.

FIG. 4 shows a schematic representation of the secondary reaction of an INVADER HIV-RNA assay.

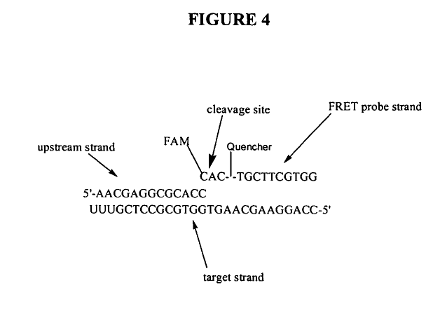

FIG. 5 shows dT-10 mer, an oligonucleotide generated using periodate salts as an oxidizing agent.

### DEFINITIONS

To facilitate an understanding of the present invention, a number of terms and phrases are defined below.

It is to be understood that unless otherwise indicated, this invention is not limited to specific reagents, reaction conditions, synthetic steps, or the like, as such may vary. It is also to be understood that the terminology used herein is for the purpose of describing particular embodiments only and is not intended to be limiting.

Furthermore, it is noted that, as used in the specification and the appended claims, the singular forms. “a,” “an” and “the” include plural referents unless the context clearly dictates otherwise. Thus, for example, reference to “a protecting group” includes combinations of protecting groups, reference to “a nucleoside” includes combinations of nucleosides, and the like. Similarly, reference to “a substituent” as in a compound substituted with “a substituent” includes the possibility of substitution with more than one substituent, wherein the substituents may be the same or different.

As used herein, the term “a reagent that specifically detects expression levels” refers to reagents used to detect the expression of one or more genes (e.g., including but not limited to, genes detectable with oligonucleotides synthesized according to methods of the present invention). Examples of suitable reagents include, but are not limited to, nucleic acid probes capable of specifically hybridizing to the gene of interest, PCR primers capable of specifically amplifying the gene of interest, and IVADER assay reagents.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxy-aminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and/or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used herein, the terms “an oligonucleotide having a nucleotide sequence encoding a gene” and “polynucleotide having a nucleotide sequence encoding a gene,” means a nucleic acid sequence comprising the coding region of a gene or in other words the nucleic acid sequence that encodes a gene product. The coding region may be present in a cDNA, genomic DNA or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the terms “complementary” or “complementarity” are used in reference to polynucleotides (i.e., a sequence of nucleotides) related by the base-pairing rules. For example, for the sequence “5′-A-G-T-3′,” is complementary to the sequence “5′-T-C-A-3′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i. e., selective) interaction. The absence of non-specific binding may be tested by the use of a second target that is substantially non-complementary (e.g., less than about 30% identity); in the absence of non-specific binding the probe will not hybridize to the second non-complementary target.

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

When used in reference to a single-stranded nucleic acid sequence, the term “substantially homologous” refers to any probe that can hybridize (i.e., it is the complement of) the single-stranded nucleic acid sequence under conditions of low stringency as described above.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.” As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization (1985)). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under ‘medium stringency conditions,” a nucleic acid sequence of interest will hybridize only to its exact complement, sequences with single base mismatches, and closely relation sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

“High stringency conditions” when used in reference to nucleic acid hybridization comprise conditions equivalent to binding or hybridization at 42° C in a solution consisting of 5× SSPE (43.8 g/l NaCl, 6.9 g/l NaH₂PO₄ H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 0.1× SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5× SSPE (43.8 g/l NaCl, 6.9 g/l NaH₂PO₄ H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 1.0× SSPE, 1.0% SDS at 42° C. when a probe of about 500 nucleotides in length is employed.

“Low stringency conditions” comprise conditions equivalent to binding or hybridization at 42° C. in a solution consisting of 5× SSPE (43.8 g/l NaCl, 6.9 g/l NaH₂PO₄ H₂O and 1.85 g/l EDTA, pH adjusted to 7.4 with NaOH), 0.1% SDS, 5× Denhardt's reagent (50× Denhardt's contains per 500 ml: 5 g Ficoll (Type 400, Pharamcia), 5 g BSA (Fraction V; Sigma)) and 100 μg/ml denatured salmon sperm DNA followed by washing in a solution comprising 5× SSPE, 0.1% SDS at 42° C. when a probe of about 500 nucleotides in length is employed.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.) (see definition above for “stringency”).

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, that is capable of hybridizing to at least a portion of another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein the term “portion” when used in reference to a nucleotide sequence (as in “a portion of a given nucleotide sequence”) refers to fragments of that sequence. The fragments may range in size from four nucleotides to the entire nucleotide sequence minus one nucleotide (10 nucleotides, 20, 30, 40, 50, 100, 200, etc.).

As used herein, the terms “restriction endonucleases” and “restriction enzymes” refer to bacterial enzymes, each of which cut double-stranded DNA at or near a specific nucleotide sequence.

The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of components (e.g., contaminants) from a sample. For example, antibodies are purified by removal of contaminating non-imniunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

The term “RNA interference” or “RNAi” refers to the silencing or decreasing of gene expression by siRNAs. It is the process of sequence-specific, post-transcriptional gene silencing in animals and plants, initiated by siRNA that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial.

It will be appreciated that, as used herein, the terms “nucleoside” and “nucleotide” will include those moieties that contain not only the known purine and pyrimidine bases, but also modified purine and pyrimidine bases and other heterocyclic bases that have been modified (these moieties are sometimes referred to herein, collectively, as “purine and pyrimidine bases and analogs thereof”). Such modifications include methylated purines or pyrimidines, acylated purines or pyrimidines, and the like.

By “protecting group” as used herein is meant a species that prevents a segment of a molecule from undergoing a specific chemical reaction, but that is removable from the molecule following completion of that reaction. This is in contrast to a “capping group,” that permanently binds to a segment of a molecule to prevent any further chemical transformation of that segment.

“Optional” or “optionally” means that the subsequently described circumstance may or may not occur, so that the description includes instances where the circumstance occurs and instances where it does not. For example, the phrase “optionally substituted” means that a non-hydrogen substituent may or may not be present, and, thus, the description includes structures wherein a non-hydrogen substituent is present and structures wherein a non-hydrogen substituent is not present.

As used herein, the terms “computer memory” and “computer memory device” refer to any storage media readable by a computer processor. Examples of computer memory include, but are not limited to, RAM, ROM, computer chips, digital video disc (DVDs), compact discs (CDs), hard disk drives (HDD), and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks.

As used herein, the terms “processor” and “central processing unit” or “CPU” are used interchangeably and refer to a device that is able to read a program from a computer memory (e.g., ROM or other computer memory) and perform a set of steps according to the program.

### DETAILED DESCRIPTION OF THE INVENTION

In 1983, a breakthrough was achieved in solid phase synthesis chemistry that made it possible to make longer and longer oligonucleotides and to make them much more efficiently. The new synthesis process was based upon the use of phosphoramidite monomers and the use of tetrazole catalysis (See, e.g., McBride and Caruthers, Tetrahedron Lett. 24:245-248.1983).

Generally, phophoramidite synthesis begins with the 3′-most nucleotide and proceeds through a series of cycles composed of fours steps that are repeated until the 5′-most nucleotide is attached. These steps are deprotection, coupling, capping, and stabilization. In the classic deprotection step the trityl group attached to the 5′ carbon of the pentose sugar of the recipient nucleotide is removed by trichloroacetic acid (TCA) leaving a reactive hydroxyl group. At this stage the next phosphoramidite monomer is added. Bemer et al. (Nucleic Acids Res 17: 853-864 (1989)) showed that tetrazole, a weak acid, attacks the coupling phosphoramidite nucleoside forming a tetrazolyl phosphoramidite intermediate. This structure then reacts with the hydroxyl group of the recipient and the 5′ to 3′ linkage is formed. The tetrazole is reconstituted and the process continues. The use of tetrazole increased coupling efficiency to greater than 99% and, with this, opened the way for longer and longer oligonucleotides to be synthesized.

While the increased efficiency afforded by the advent of tetrazole phosphoramidite intermediate coupling was a major advance in oligonucleotide synthesis, it was still a chemical process and there was a finite failure rate. A coupling failure results in an oligonucleotide still having a reactive hydroxyl group on its 5′-most end. If this were to remain freely reactive, it would be able to couple in the next round and the result would be a missing base in the synthesis. Thus, coupling failures had to be removed from further participation in the synthesis. In general, this is accomplished by adding capping agents (e.g., an acetylating reagent composed of acetic anhydride and N-methyl imidazole). Capping reagents react only with free hydroxyl groups to irreversibly cap the oligonucleotides in which coupling failed.

Once the capping step is accomplished the last step in the cycle is oxidation which stabilizes the phosphate linkage between the growing oligonucleotide chain and the most recently added base. Generally, this step has been carried out in the presence of iodine (e.g., as a mild oxidant) in tetrahydrofuran (THF) and water. The oxidation step is important for successful oligonucleotide synthesis, as it permits the acid promoted removal of the protecting groups and subsequent repetition of the oligonucleotide synthesis cycle.

Thus, following this final step the cycle is repeated for each nucleotide in the sequence. At the end of the synthesis the oligonucleotide exists with the 3′ end still attached to a solid support (e.g., a controlled pore glass bead (CPG)) and the 5′ end protected with a trityl group. Generally, in addition, there are protecting groups on three of the four bases. These can be present to maintain the integrity of the ring structures of the bases. The protecting groups may be benzoyl on A and C and N-2-isobutyryl on G. Thymidine needs no protecting group. The completed synthesis is cleaved off the CPG and then detritylated leaving a hydroxyl on both the 3′ and 5′ ends. At this point the oligonucleotide is deprotected and exists as a functional single-stranded DNA molecule. Deprotection removes the protecting groups, but they remain with the oligonucleotide as organic salts that must be removed. The process of removing these contaminants is called desalting.

During the last two decades, each of the four synthetic steps of the phosphoramidite protocol (detritylation, coupling, oxidation and capping) have been studied extensively and new reagents or new procedures bringing improvement or modifications into synthetic steps have been reported (See, e.g., Reese and Yan, Tetrahedron Letters, 2004, 45, 2567-2570; Krotz et al., Organic Process Research & Development 2003, 7, 47-52; Habus and Agrawal, Nucleic Acids Research, 1994, 22, 4350-4351; Ohkubo et al., Tetrahedron Letters 2004, 45, 363-366; Sekine et al., J. Org. Chem. 2003, 68, 5478-5492; Eleuteri et al., Organic Process Research & Development 2000, 4, 182-189; Marshall et al.,Kaiser, Current Opinion in Chemical Biology 2004, 8, 222-229; Sanghvi et al., Organic Process Research & Development 2000, 4, 175-181; Gao et al., Biopolymers 2004, 73, 579-596; Kumar et al., J. Org. Chem. 2004, 69, 6482). Among those synthetic steps, oxidation is one of the most important. This step converts the acid sensitive internucleotide phosphite triester linkage into a stable phosphate linkage, which makes possible the acid promoted removal of the 5′-DMT protecting group and subsequent coupling with the next appropriate phosphoramidite reagent.

As stated above, most phosphoramidite coupling protocols utilize iodine in THF/water/pyridine solution as the reagent of choice for oxidation of the unstable phosphite group. In spite of its popularity, the use of this reagent often leads to unwanted side effects, particularly in cases when water, base, or iodine sensitive groups (e.g., linkers or fluorescent dyes, labels, or other detectable groups) are present in the structure of the chemically synthesized oligonucleotide molecule.

The present invention provides novel reagents (e.g., periodate salts) that are compatible with solid phase phosphoramidite oligonucleotide synthesis. In preferred embodiments, the reagents are compatible with (e.g., are stable and soluble in) organic solvents. In addition to their use under non-aqueous conditions, the compositions and methods of the present invention are easily obtainable and inexpensive. Further, because the reagents find use with fluorescent or other readily detectable protecting groups, the invention provides compositions and methods enabling monitoring of individual reaction steps of solid phase synthesis. The present invention also readily lends itself to the highly parallel, microscale synthesis of oligonucleotides (e.g., for generating microarrays). Furthermore, in some embodiments, the present invention finds use in solution phase synthesis of oligonucleotides (e.g., for generation of oligonucleotides for use in RNAi applications, See, e.g., U.S. Pat. App. No. 20040116685, herein incorporated by reference).

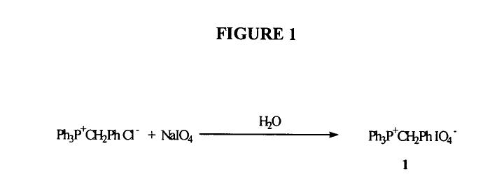

Specifically, the present invention provides that a periodate salt (e.g., a periodate salt and/or solution in anhydrous organic solvent) can be used as an efficient oxidizer converting the labile phosphite bond into the more stable internucleotide phosphate bond in the phosphoramidite oligonucleotide synthesis process described herein (See, e.g., Examples 1, 2, and 3). In some embodiments, the organic solvent comprises methylene chloride. In other embodiments, the organic solvent comprises acetonitrile. The present invention is not limited to any particular periodate salt or solution. Indeed a variety of periodate salts and solutions are contemplated to be useful in the present invention including, but not limited to, tetrabutylammonium periodate and phosphonium periodate. In some embodiments, the phosphonium periodate is benzyltriphenylphosphonium periodate 1. In some embodiments, benzyltriphenylphosphonium periodate 1 is generated according to the methods of Hajipour et al., 2001, Synlett, 11, 1735-1738 (See Example 1 and FIG. 1). In some embodiments, the periodate salt is a sodium periodate or a tetraalkylammonium periodate. The present invention is not limited to any solvent (e.g., anhydrous solution into which a periodate salt of the present invention is solubilized). Indeed, a variety of solvents can be utilized including, but not limited to, acetonitrile, acetone, and methylene chloride. Similarly, the present invention is not limited to any particular concentration of periodate salt utilized (e.g., in an oligonucleotide synthesis reaction described herein). For example, in some embodiments, the periodate salt concentration may from about 0.01 M to about 0.3 M, and in some embodiments from about 0.1 M to about 0.15 M, although concentrations above 0.3 M and less than 0.01 M may be used.

The present invention is not limited to phosphoramidite coupling chemistry, but is compatible with other coupling reactions (e.g., H-phosphonate or phosphate triester coupling chemistry). The present invention also lends itself to automated oligonucleotide synthesis and is ideally suited for the large scale manufacture of oligonucleotides with high efficiency.

The present invention demonstrates that a composition comprising a periodate salt is milder compared to the iodine-based oxidizing reagent traditionally used in solid phase oligonucleotide synthesis. Thus, in preferred embodiments, the present invention provides a phosphoramidite coupling protocol utilizing a periodate salt (e.g., a phosphonium periodate) as the reagent of choice for oxidation of the unstable phosphite group present during each cycle of oligonucleotide synthesis using phosphoramidite chemistry. The present invention discloses that uses of periodate in an oligonucleotide synthesis scheme can be just as efficient as traditional reagents (e.g., iodine solutions) (See, e.g., Example 2). Furthermore, the present invention provides reagents (e.g., periodate salts) compatible with water, base and iodine sensitive groups present in chemically synthesized oligonucleotide molecules (e.g., with fluorescent or other readily detectable protecting groups).

Thus, the present invention provides methods for synthesizing an oligonucleotide (e.g., on a solid support) wherein an oxidizing reagent comprising a periodate salt (e.g., in an organic solvent) is used during the oxidation step of synthesis. Although an understanding of the mechanism is not necessary to practice the present invention and the present invention is not limited to any particular mechanism of action, in some embodiments, use of an oxidizing reagent comprising a periodate salt stabilizes the phosphate linkage between the growing oligonucleotide chain and the most recently added base. In some embodiments, the presence of a periodate salt of the present invention functions as an oxidant. Although an understanding of the mechanism is not necessary to practice the present invention and the present invention is not limited to any particular mechanism of action, in some embodiments, the presence of a periodate salt (e.g., in an organic solvent) converts the acid sensitive internucleotide phosphite triester linkage into a stable phosphate linkage. Thus, in some embodiments, the present invention provides the stabilization of phosphate linkages thereby enabling the acid promoted removal of the 5′-DMT protecting group (e.g., it is contemplated that with more stable/robust phosphate linkage a stronger acid can be used to remove protecting groups) and subsequent coupling with the appropriate phosphoramidite reagent (e.g., in a subsequent synthesis cycle). Thus, methods of the present invention provide improved oligonucleotide synthesis efficiency as well as an improved capability to synthesize oligonucleotides comprising reagents sensitive to previous synthesis chemistries (e.g., sensitive to water and iodine). For example, in some embodiments, the present invention can be utilized to generate oligonucleotides comprising azido molecules or acyl phosphate (See, e.g., WO 03/079014 and U.S. Pat. App. No. 20050043507, respectively, each of which is herein incorporated by reference in its entirety).

In some embodiments, a nucleoside may be bound to a support through its 3′-hydroxyl group or its 5′-hydroxyl group. A second nucleoside monomer is then coupled to the free hydroxyl group of the support-bound initial monomer, wherein for 3′-to-5′ oligonucleotide synthesis, the second nucleoside monomer has a phosphorus derivative such as a phosphoramidite at the 3′ position and a protecting group at the 5′ position, and alternatively, for 5′-to-3′ oligonucleotide synthesis, the second nucleoside monomer has a phosphorus derivative at the 5′ position and a protecting group at the 3′ position. This coupling reaction gives rise to a newly formed phosphite triester bond between the initial nucleoside monomer and the added monomer, with ,the protected hydroxyl group intact. In preferred embodiments, an oxidizing reagent comprising a periodate salt stabilizes the phosphate linkage between the growing oligonucleotide chain and the most recently added nucleoside base.

In some embodiments, it is contemplated that any solid support may serve as the starting point for oligonucleotide synthesis. For example, the synthetic methods of the invention may be conducted on any solid substrate having a surface to which chemical entities may bind. Suitable solid supports are typically polymeric, and may have a variety of forms and compositions and derive from naturally occurring materials, naturally occurring materials that have been synthetically modified, or synthetic materials. Examples of suitable support materials include, but are not limited to, polysaccharides such as agarose (e.g., that available commercially as SEPHAROSE, from Pharmacia) and dextran (e.g., those available commercially under the tradenames SEPHADEX and SEPHACYL, also from Pharmacia), polyacrylamides, polystyrenes, polyvinyl alcohols, PEG polymers, copolymers of hydroxyethyl methacrylate and methyl methacrylate, silicas, teflons, glasses, and the like. In some embodiments, the initial monomer of the oligonucleotide to be synthesized on the substrate surface is bound to a linking moiety. In some embodiments, the linking moiety is bound to a surface hydrophilic group (e.g., to a surface hydroxyl moiety present on a silica substrate).

### EXPERIMENTAL

The following examples are provided in order to demonstrate and further illustrate certain preferred embodiments and aspects of the present invention and are not to be construed as limiting the scope thereof.

In the experimental disclosure that follows, the following abbreviations apply: ° C. (degrees Centigrade); cm (centimeters); g (grams); l or L (liters); μg (micrograms); μl (microliters); μm (micrometers); μM (micromolar); μmol (micromoles); mg (milligrams); ml (milliliters); mm (millimeters); mM (millimolar); mmol (millimoles); M (molar); mol (moles); ng (nanograms); nm (nanometers); nmol (nanomoles); N (normal); and pmol (picomoles).

### Example 1

### Preparation of Phosphonium Periodate 1

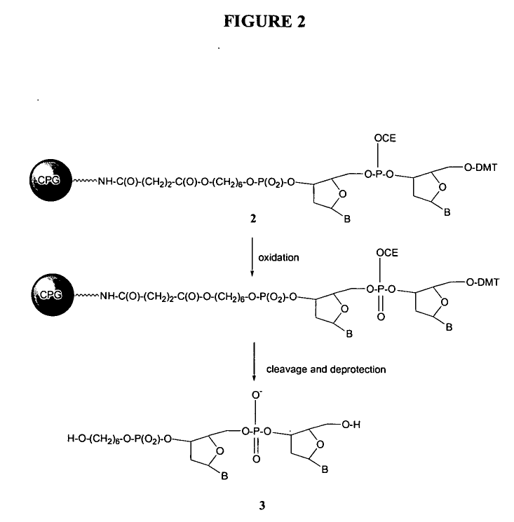

During development of the present invention, experiments were aimed at comparing the efficiency of oxidation of the dinucleotide phosphite bond in material 2 (See, e.g., FIG. 2) by a solution of sodium periodate in DMF, versus a solution of phosphonium periodate 1 (See, e.g., FIG. 1) in acetonitrile, versus a solution of tetrabutylammonium periodate (e.g., commercially available from Aldrich) in acetonitrile, as well as the oxidation via the conventionally used iodine-based oxidizing reagent (0.02 M I₂/water/THF/Pyridine) (See, e.g., FIGS. 1 and 2).

Generally, the preparation of material 2 used in the experiments was accomplished by coupling of the deoxynucleotide phosphoramidite to DMT protected hexanediol CPG, oxidation of the intermediate phosphite bond by the conventional iodine-based oxidizer, removal of the 5′-DMT protecting group and subsequent coupling with the appropriate deoxynucleotide phosphorarnidite. The last 5′-DMT protecting group was preserved. Although the present invention contemplates the use of any nucleotide synthesizer, all of the above steps were performed using the ABI 8909 synthesizer and standard phosphoramidite coupling protocol.

After the completion of the synthesis, material 2 was removed from the cartridge and transferred into a 2 ml Hamilton gas-tight syringe fitted at the inlet with a plug of glass wool. To minimize the interaction of material 2 with atmospheric oxygen, cartridges containing material 2 were stored in acetonitrile and were quickly dried with argon directly before transfer into the syringe. To complete the oxidation step leading to the preparation of compound 3, the solution of the oxidizer was drawn into the syringe and incubated with material 2 for a specific period of time. In these experiments, the solid phase suspended dinucleotide phosphites 2 were exposed to the solution of the oxidizing reagent for 1 min, 5 min and 1 hour respectively, and then subsequently washed with dry acetonitrile (6×2 ml).

After removal of the 5′-DMT protecting group with TCA solution in dichloromethane (DNA synthesis grade reagent), the material was washed with acetonitrile/pyridine 1:(1×2 ml) and acetonitrile (6×2 ml). Finally, the resulting reaction product was transferred into a screw-cap vial. Cleavage and deprotection was carried out using concentrated ammonia at 55° C. All crude reaction products were analyzed by C¹⁸ RP-HPLC. As expected, the use of the iodine-based oxidizer resulted in the formation of fully oxidized dinucleotide 3 (B=A, C, G, T) under all of the above reaction conditions.

In comparison to the iodine-based reagent, it was found that 0.1 M sodium periodate in DMF, 0.15M phosphonium periodate 1 in acetonitrile and 0.15M tetrabutylammonium periodate in acetonitrile were capable of full conversion of the internucleotide phosphite bond into the stable internucleotide phosphate bond, although at these concentrations were somewhat less efficient (e.g., required longer oxidation times). It was found that in order to achieve full oxidation of the internucleotide phosphite bond in the material 2, 20 min oxidation time was required in the case of 0.1 M DMF solution of sodium periodate. However, the 0.15M acetonitrile solutions of the phosphonium periodate 1 and tetrabutylammonium periodate represented more efficient oxidizers. Only 5 min oxidation time was required for formation of the dinucleotides 3 (B=A, C, G, T) which were chromatographically identical with the dinucleotides 3 synthesized using conventional iodine-based oxidizing reagent.

The search for better oxidation reaction conditions required testing the influence of other solvents used to dissolve the periodate 1 on the speed of the oxidation of the phosphite bond in compound 2. In test experiments, the material 3 produced by treatment of the phosphite 2 (B=T) for 1 min was compared with the 0.15M solution of periodate 1 in acetonitrile, acetone and methylene chloride. Since the HPLC analysis of the crude oxidation products 3 did not reveal any substantial differences between the materials generated using solution of the periodate 1 in different solvents during 1 min oxidation time, acetonitrile was used as a solvent of choice for further experiments. It was also encouraging to find that the RP HPLC analysis of the dinucleotides 3 (B=A, C, G, T) synthesized by treatment of the phosphates 2 for 1 hour at room temperature with the 0.15M acetonitrile solution of the periodate 1 did not show any changes or differences in the material composition when compared to the HPLC profiles of dinucleotides 3 synthesized using standard synthetic protocol. The present invention therefore provides that either the acetonitrile solution of the periodate 1 or the acetonitrile solution of tetrabutylammonium periodate can be used as a novel reagent in oligonucleotide synthesis (e.g., automated synthesis) leading to the oxidation of the internucleotide bond under nonbasic, anhydrous reaction conditions. As a test for the compatibility of the acetonitrile solution of phosphonium periodate 1 and the acetonitrile solution of tetrabutylammonium periodate with automated oligonucleotide synthesis, automated synthesis of the dT-10 mer (compound 4) was performed using the above oxidizing reagents (See Example 2, below).

### Example 2

### Synthesis of a dT-10 mer using Periodate Salts

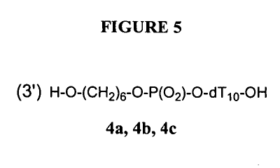

The synthesis of compound 4 (See, e.g., FIG. 5) was performed using an ABI 8909 synthesizer and applying a standard synthetic protocol (e.g., with iodine as the oxidizing agent) for solid phase phosphoramidite oligonucleotide synthesis (synthesis of compound 4a). The synthesis of oligonucleotide 4 was also performed using modified synthetic protocols in which the iodine-based oxidizer was replaced by 0.15 M acetonitrile solutions of the phosphonium periodate 1 (synthesis of compound 4b) and tetrabutylammonium periodate (synthesis of compound 4c) and increasing the time of the oxidation step to 7 min. Initial experiments of the oxidation of phosphite 2 performed manually in the syringe indicated that 5 min oxidation time was sufficient to fully oxidize the internucleotide phosphite bond in the material 2. However, it was found that the extension of the oxidation time to 7 min while performing the oxidation step on the DNA synthesizer was needed in order to achieve full oxidation and final material of higher quality.

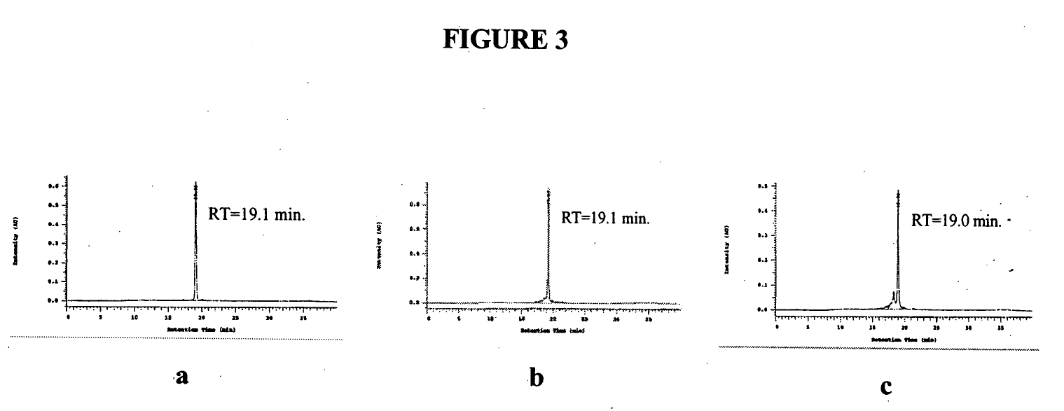

After all synthetic steps were completed, the synthesized oligonucleotides were detached from the solid support by treatment with concentrated ammonia giving compound 4a synthesized using conventional synthetic protocol, compound 4b synthesized with the use of the periodate 1 as oxidizing reagent and compound 4c synthesized with the use of the tetrabutylammonium periodate as the oxidizing reagent. The comparison of the RP HPLC profiles (See FIG. 3) of compounds 4a, 4b and 4c indicated the formation of the desired material in each case. The structural identity of compounds 4a, 4b and 4c was additionally confirmed by MALDI-TOF analysis. The kP HPLC profiles of the crude products 4b and 4c demonstrated also that acetonitrile phosphonium periodate 1 represents a more efficient oxidixing reagent compared to the acetonitrile solution of tetrabutylammonium periodate. The use of the periodate 1 resulted in the preparation of material 4b with the efficiency comparable to those achieved when the standard iodine-based oxidizer was used (preparation of compound 4a) (See FIG. 3). As an additional test for the applicability of solution periodate 1 in the synthesis of functional mixed-base DNA probes, DNA probe 5 was synthesized and used as an upstream strand in an INVADER assay. (See Example 3, below).

### Example 3

### Synthesis of DNA Probes using Periodate Salts and Applications using the Same

DNA probes 5 (5′-AACGAGGCGCACC-3′ (SEQ ID NO. 1), upstream strand) were synthesized using standard automated phosphoramidite coupling protocol (material 5a) and a modified protocol utilizing 0.15 M solution of phosphonium periodate 1 in acetonitrile (material 5b) with 7 min oxidation time. After cleavage from the solid support, deprotection using concentrated ammonia (55° C./16 hr) and IE HPLC purification, the identity of both probes was confirmed by MALDI-TOF analysis. Subsequently, probes 5a and 5b were used as upstream strands in an INVADER assay (See INVADER assays, Third Wave Technologies; See e.g., U.S. Pat. Nos. 5,846,717; 6,090,543; 6,001,567; 5,985,557; 6,090,543; 5,994,069, 6,348,314, 6,692,917, 6,555,387; Lyamichev et al., Nat. Biotech., 17:292 (1999), Hall et al., PNAS, USA, 97:8272 (2000), WO97/27214 and WO98/42873, each of which is herein incorporated by reference in its entirety for all purposes).

Briefly, assays were performed using 10 μl reaction samples containing 2 μM probe, 2 μM target and 10 nM upstream oligonucleotides 5a or 5b with 256 nM CLEAVASE enzyme AfuFEN. CLEAVASE enzyme AfuFEN was stored in 50% glycerol, 20 mM Tris-HCl, pH 8, 50 mM KCl, 0.5% Tween-20, 0.5% Nonidet-P40, 100 μg/ml BSA. The reaction buffer contained 1.4% PEG, 4 mM MOPS, 5.6 mM MgCl₂, 0.002% PROCLIN and the reaction samples were incubated for 15 minutes at 63° C. in a PTC-100 (MJ Research). The FRET probe strand contains fluorescein as a reporter moleule (6-FAM, Glen Research) and ECLIPSE Quencher (Epoch ECLIPSE, Glen Research) as a quenching molecule. Assay plates were analyzed with a SAFIRE platereader (Tecan), settings for FAM dye detection (wavelength/bandwith) were: excitation: 485/5 nm; emission 520/5 nm.

The degree of the invasive cleavage leading to the generation of the fluorescent, dye labeled DNA fragment 5′-FAM-C was monitored by a SAFIRE fluorescence plate reader (Tecan). Fold-over zero values (FOZ) were determined by calculating the ratio of the fluorescence signal from samples containing the upstream strand and the no upstream strand control. Table 1 below shows the efficiency of the invasive cleavage of the INVADER assay observed for the probes 5a and 5b.

TABLE 1

| Upstream strand | FOZ |
|---|---|
| Probe 5a | 13.1 |
| Probe 5b | 13.5 |

The comparable results of the INVADER assay generated for compound 5a and 5b confirmed the structural authenticity of probes synthesized using both standard synthetic protocol and protocol utilizing the solution of periodate 1 as an oxidizing reagent.

All publications and patents mentioned in the above specification are herein incorporated by reference. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

What is claimed is:

1. A method for synthesizing oligonucleotides, comprising stabilizing a phosphate linkage between a growing oligonucleotide chain and most recently added base with a reagent comprising a periodate salt.

2. The method of claim 1, wherein said stabilizing comprises converting an acid sensitive internucleotide phosphite triester linkage into a stable phosphate linkage.

3. The method of claim 1, further comprising:
a) removing a protecting group from a 5′ or 3′ carbon of the pentose sugar of a recipient nucleotide; thereby leaving a reactive hydroxyl;
b) coupling a phosphoramidite monomer to said reactive hydroxyl; and
c) capping unreacted hydroxyls.

4. The method of claim 3, wherein said protecting group comprises a trityl group.

5. The method of claim 4, wherein said trityl group is dimethoxytrityl.

6. The method of claim 1, wherein said periodate salt is present within an organic solvent.

7. The method of claim 3, wherein said protecting group is removed via exposure to acid.

8. The method of claim 7, wherein said acid is trichloroacetic acid (TCA).

9. The method of claim 1, wherein said periodate salt comprises tetrabutylammonium periodate.

10. The method of claim 1, wherein said periodate salt comprises phosphonium periodate.

11. The method of claim 10, wherein said phosphonium periodate is benzyltriphenylphosphonium periodate.

12. The method of claim 1, wherein said periodate salt is soluble within an organic solvent.

13. The method of claim 6, wherein said organic solvent comprises methylene chloride.

14. The method of claim 6, wherein said organic solvent comprises acetonitrile.

15. The method of claim 6, wherein said organic solvent comprises acetone.

16. The method of claim 1, wherein said oligonucleotides comprise a detectable label.

17. The method of claim 16, wherein said detectable label comprises a fluorescent molecule.

18. The method of claim 17, wherein said fluorescent molecule comprises fluorescein.

19. The method of claim 1, wherein said synthesizing oligonucleotides comprises solid phase synthesis.

20. The method of claim 19, wherein said solid phase synthesis comprises a nucleoside bound to a support through its 5′-hydroxyl group.

21. A method for preparing an oligonucleotide for acid-based removal of a protecting group comprising treating said oligonucleotide with a reagent comprising a periodate salt.

22. The method of claim 21, wherein said reagent comprising a periodate salt converts an acid sensitive internucleotide phosphite triester linkage into a stable phosphate linkage.

23. The method of claim 21, wherein said reagent comprising a periodate salt is applied during each cycle of oligonucleotide synthesis.

24. The method of claim 23, wherein said periodate salt is selected from the group consisting of tetrabutylammonium periodate, phosphonium periodate, and benzyltriphenylphosphonium periodate.

25. A composition comprising:
a) a periodate salt; and
b) a nucleotide.

26. The composition of claim 25, further comprising a reagent for nucleic acid synthesis selected from the group consisting of phosphoramidite monomer, tetrazol, an acid, a linker, a label, a dye, a solid support, a protecting group, and a microarray plate.
